(19) United States
(12) Patent Application Publication
(10) Pub. No.: US 2010/0003278 A1
(43) Pub. Date: Jan. 7, 2010

(54) Immunological Compositions Effective for Lessening the Severity or Incidence of PRRSV Signs and Methods of Use Thereof

(75) Inventors: Michael Roof, Ames, IA (US); Eric Vaughn, Ames, IA (US); Wesley Johnson, Ames, IA (US)

Correspondence Address: Michael P. Morris;Boehringer Ingelheim USA Corporation, 900 Ridgebury Road, Ridgefield, CT, 06877-0368, US

(73) Assignee: BOEHRINGER INGELHEIM VETMEDICA, INC., Saint Joseph, MO (US)

(21) Appl. No.: 12/492,334

(22) Filed: Jun. 26, 2009

Related U.S. Application Data
(60) Provisional application No. 61/078,320, filed on Jul. 3, 2008.
(60) Provisional application No. 61/089,882, filed on Aug. 18, 2008.

(51) Int. Cl. A61K 39/12; A61P 31/12

(52) U.S. Cl. 424/204.1

(57) ABSTRACT

The present application describes improved an immunogenic compositions of virus vaccines wherein the virus vaccines comprise adjuvants selected from the group consisting of MCP-1, Haemophilus sonmus fractions, carbomer and combinations thereof. Methods and compositions using such improved compositions are described.

### RELATED APPLICATIONS

The present application claims the benefit of priority of U.S. Provisional Application No. 61/078,320 which was filed on Jul. 3, 2008 and of U.S. Provisional Application No. 61/089,882 which was filed Aug. 18, 2008. The entire text of each of the aforementioned applications is incorporated herein by reference.

### BACKGROUND OF THE INVENTION

Porcine Reproductive and Respiratory Syndrome (PRRS) is a disease that has been described in North America and Europe. The disease is also known as Swine Infertility and Respiratory Syndrome (SIRS) and Porcine Epidemic Abortion and Respiratory Syndrome (PEARS). Infected farms may experience severe reproductive losses, including spontaneous abortions, the birth of stillborn pigs and mummified fetuses, increased incidence of pneumonia, and poor growth rate of pigs from the nursery through the finishing stages.

Human macrophage/monocyte chemotactic and activating factor (MCAF) also called human macrophage chemoattractant protein-1 (MCP-1) (newly named CCL-2) is an 8.6 kDa protein containing 76 amino acid residues. MCP-1 has also been called small inducible cytokine A2 (SCYA2) and monocyte chemotactic and activating factor (MCAF). Monocyte chemotactic protein-1, a member of the small inducible gene (SIG) family, plays a role in the recruitment of monocytes to sites of injury and infection. The gene for MCP-1 is on chromosome 17 in region 17q11.2-q12.

MCP1 has been found in the joints of people with rheumatoid arthritis where may serve to recruit macrophages and perpetuate the inflammation in the joints. MPC-1 has also been found elevated in the urine of people with lupus as a sign warning of inflammation of the kidney. MCP-1 has been implicated in promoting migration of monocytes into the arterial intima. Emerging evidence suggests that an inflammatory process is involved in the pathogenesis of intractable cardiovascular diseases, including restenosis, atherosclerotic complications resulting from plaque rupture, severe tissue ischemia, and heart failure. In particular, inflammatory responses to arterial injury, which cause continuous recruitment and activation of monocytes mainly through activation of the monocyte chemoattractant protein-1 (MCP-1) pathway, have a central role in restenosis and atherogenesis.

Inflammatory changes in the arterial wall have a central role in the development of restenosis and atherosclerosis. Various mediators such as adhesion molecules, cytokines, and chemokines are involved in the initiation and progression of atherosclerotic lesions. Monocyte chemoattractant protein-1 (MCP-1) is the most important chemokine that regulates migration and infiltration of monocytes/macrophages. The effects of MCP-1 are mediated mainly through CC chemokine receptor 2 (CCR2). MCP-1 causes chronic vascular inflammation and induces thrombosis, proliferation and migration of vascular smooth muscle cells, angiogenesis, and oxidative stress. Previous studies indicate that MCP-1 production from endothelial cells, smooth muscle cells, and lesional leukocytes increases in the presence of endothelial dysfunction and atherosclerotic risk factors.

A glycosylated natural form of MCP-1/JE of 30 kDa has been found to be secreted into the conditioned medium by a medullary-type murine thymic epithelial cell line (MTEC1). The 30 kDa glycosylated form of MCP-1 shows lower specific chemotactic activity for both lymphocytes and monocytes than the 6-7 kDa unglycosylated form of MCP-1.

MCP-1 is expressed by monocytes, vascular endothelial cells, smooth muscle cells, glomerular mesangial cell, osteoblastic cells, and human pulmonary type-2-like epithelial cells in culture. It is constitutively produced by the human glioma U-105MG cell line. MCP1 mRNA is induced in human peripheral blood mononuclear leukocytes by phytohemagglutinin (PHA), bacterial lipopolysaccharides, and IL1, but not by IL2, TNF, or IFN-gamma. In mesangial cells the synthesis and release of MCP-1 is rapidly induced by IgG complexes, but not monomeric IgG or F(ab′)2 fragments of IgG.

MCP-1 is chemotactic for monocytes but not neutrophils. Maximal induction of migration is observed at a concentration of 10 ng/ml. Point mutations have been described at two amino acid positions which alter the factor so that it is then also chemotactic for neutrophils.

Elevated levels of MCP-1 are observed in macrophage-rich atherosclerotic plaques. The factor activates the tumoricidal activity of monocytes and macrophages in vivo. It regulates the expression of cell surface antigens (CD11c, CD11b) and the expression of cytokines (IL1, IL6). MCP-1 is a potent activator of human basophils, inducing the degranulation and the release of histamines. In basophils activated by IL3, IL5 or GM-CSF MCP-1 enhances the synthesis of leukotriene C4.

IL1, TNF-alpha, PDGF, TGF-beta, and LIF induce the synthesis of MCP-1 in human articular chondrocytes, which may thus play an active role in the initiation and progression of degenerative and inflammatory arthropathies by promoting monocyte influx and activation in synovial joints.

MCP-1 has been shown to exhibit biological activities other than Chemotaxis. It can induce the proliferation and activation of killer cells known as CHAK (CC-Chemokine-activated killer), which are similar to cells activated by IL2.

Two MCP-1-specific receptors have been cloned which signal in response to nanomolar concentrations of MCP-1. The two receptors differ in their carboxyl tails as a result of alternative splicing. They are related closely to the receptor for the Chemokines MIP-1-alpha and RANTES. MCP-1 also binds to a receptor designated D6. MCP-1 also binds to CCR10. MCP-1 has been shown to bind to the virus-encoded viroceptor M3. The orphan receptor L-CCR has been suggested to function as an MCP-1 receptor in glial cells.

Monocyte chemoattractant protein-1 (MCP-1) attracts monocytes bearing the chemokine receptor CCR-2. Monocyte chemoattractant protein (MCP)-1 (CCL2) specifically attracts monocytes and memory T cells. Its expression occurs in a variety of diseases characterized by mononuclear cell infiltration, and there is substantial biological and genetic evidence for its essential role in atherosclerosis and multiple sclerosis. Despite intensive screening, there are as yet no small-molecule antagonists of the receptor of MCP-1/CCL2, CCR2. Recent evidence from genetically modified mice indicates that MCP-1 and CCR2 have unanticipated effects on T helper (Th) cell development. However, unlike the identical phenotypes of MCP-1/CCL2(−/−) and CCR2(−/−) mice in inflammatory diseases, the phenotypes of these mice are disparate in adaptive immunity: MCP-1 stimulates Th2 polarization, whereas CCR2 activation stimulates Th1 polarization.

### BRIEF SUMMARY OF THE INVENTION

The invention discloses an immunogenic composition which includes a virus, preferably PRRSV, and an adjuvant. Preferably, the adjuvant is selected from the group consisting of MCP-1, Haemophilus sonmus fractions, and combinations thereof. Several fractions of Haemophilus somnus (Hs), MCP-1, and various other adjuvant materials, in combination with Ingelvac® PRRS MLV were evaluated for potential clinical or immunological improvements in vivo when compared to the vaccine alone or challenge controls.

One aspect of the present invention includes an immunogenic composition comprising a bacterium and MCP-1. The MCP-1 is used as an adjuvant.

One aspect of the present invention includes an immunogenic composition comprising a modified live virus and MCP-1.

An additional aspect of the invention includes the immunogenic composition above, wherein the modified live virus is a PRRSV virus.

A further aspect of the invention includes an immunogenic composition comprising a virus and an adjuvant selected from the group consisting of MCP-1, Haemophilus somnus fragments, a carbomer, and combinations thereof.

Another aspect of the present invention includes the immunogenic composition above wherein the virus is a modified live PRRSV.

Another aspect of the present invention includes the immunogenic composition described above, wherein the carbomer is Carbopol.

A related aspect of the invention includes the immunogenic composition above, wherein the fractions of Haemophilus somnus are selected from the group consisting of whole cell pellet, whole cell supernate, lysed cell pellet, lysed cell supernate, membranes, and combinations thereof.

An additional aspect of the present invention includes an immunogenic composition comprising PRRSV and an adjuvant selected from the group consisting of HS, ORF 5, INF alpha, Poly ICLC, IL-12, and combinations thereof.

Another aspect of the present invention includes any of the immunogenic compositions disclosed above further comprising Carbopol as an adjuvant.

Another aspect of the present invention includes an immunogenic composition comprising a modified live PRRSV and a carbomer, preferably Carbopol.

A further aspect of the present invention includes a method for lessening the severity of clinical symptoms associated with PRRSV infection comprising the step of administering any of the immunogenic compositions listed above.

An additional aspect of the present invention includes the method above, wherein the clinical symptoms are selected from the group consisting of lung lesions, anorexia, skin discolorations, lethargy, respiratory signs, mummified piglets, coughing, diarrhea and combinations thereof.

Another aspect of the present invention includes a method for reducing the percentage of lung lesions by at least 50% when compared to animals not receiving the immunogenic composition, comprising the step of administering any of the immunogenic compositions discussed above.

A further aspect of the present invention includes a method for reducing viremia in animals by at least 45% when compared to animals not receiving the immunogenic composition including the step of administering any of the immunogenic compositions discussed above.

Another aspect of the present invention includes any of the immunogenic compositions or methods discussed above, wherein the immunogenic composition is administered by intramuscular injection.

An additional aspect of the present invention includes any of the immunogenic compositions or methods discussed above, wherein the immunogenic composition is administered in at least a 2 mL dose.

### BRIEF DESCRIPTION OF SEVERAL VIEWS OF THE DRAWINGS

FIG. 1: Average Daily Clinical Scores.

FIG. 2 Average Post-inoculation Rectal Temperatures.

FIG. 3: Post-inoculation Lung Scores.

FIG. 4: PRRS ELISA Results of Post-inoculation Serological Status.

FIG. 5: IDEXX PRRS ELISA (S/P ratio—cut-off of 0.4).

FIG. 6 : Percent positive virus isolation.

FIG. 7: Average daily clinical score (normal=3).

FIG. 8: Average gross lung pathology.

FIG. 9: IHC lesion scores.

FIG. 10: INF gamma response (ELI-SPOT).

FIG. 11: INF alpha stimulation using TGE.

FIG. 12: INF alpha stimulation using CpG.

### DETAILED DESCRIPTION OF THE INVENTION

An “immunogenic or immunological composition” refers to a composition of matter that comprises at least one antigen, which elicits an immunological response in the host of a cellular and/or antibody-mediated immune response to the composition or vaccine of interest. Usually, an “immunological response” includes but is not limited to one or more of the following effects: the production or activation of antibodies, B cells, helper T cells, suppressor T cells, and/or cytotoxic T cells and/or gamma-delta T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction or lack of clinical signs normally displayed by an infected host, a quicker recovery time and/or a lowered duration or bacterial titer in the tissues or body fluids or excretions of the infected host.

“Lung Pathology Assessment” refers to observation of the lungs after necropsy, including, but not limited to, assessment of consolidation, lesions, and nodular formations as well as assessment of the thoracic cavity including pleuritis and fluid accumulation.

The term “clinical symptoms” refers to any abnormal clinical manifestation in an animal as compared to an healthy animal.

Thus, the term “reduction of clinical symptoms” shall mean, but not limited to the reduction of any of the clinical symptoms listed herein.

The invention provides improved vaccine compositions, in particular, improved PRRS virus vaccines. Such improvements comprise preparation of the vaccines in combination with adjuvants that have been advantageously found to enhance the efficacy of the vaccine such that a better clinical response/outcome is seen with the administration of the combination of the adjuvant and the vaccine as compared to administration of the vaccine alone. In specific preferred embodiments, the immunogenic composition comprise a virus vaccine and an adjuvant selected from the group consisting of MCP-1, Haemophilus sonmus fractions, carbapol and combinations thereof. In particular, the virus vaccine is a PRRS virus vaccine, which may be a recombinant subunit vaccine or alternatively may be a live attenuated virus vaccine. An exemplary live vaccine is Ingelvac® PRRS MLV.

In addition to the above, the immunogenic compositions of the invention may contain other ingredients so long as the other ingredients do not interfere with the adjuvant properties of the MCP-1, Haemophilus sonmus fractions, carbapol or the underlying virus vaccine. Such other ingredients include, for example, binders, colorants, desiccants, antiseptics, wetting agents, stabilizers, excipients, adhesives, plasticizers, tackifiers, thickeners, patch materials, ointment bases, keratin removers, basic substances, absorption promoters, fatty acids, fatty acid ester, higher alcohols, surfactants, water, and buffer agents. Preferred other ingredients include buffer agents, ointment bases, fatty acids, antiseptics, basic substances, or surfactants.

The content or amount of the adjuvants used in the invention may be vary and can be determined by taking into consideration, for example, the properties of the PRRS virus vaccine being used, and the dosage form. The adjuvant may comprise, for example, 1 to 100% by weight. The immunogenic compositions of the invention are produced by mixing together the adjuvant component and the virus vaccine component, either alone or with various other ingredients. The compositions may be such that the virus vaccine and the adjuvant are presented as one formulation or alternatively, the adjuvant and the vaccine are presented in distinct formulations that can be administered simultaneously or sequentially.

The adjuvant component of the immunogenic compositions of the invention thus may be administered separately from the virus vaccine in the administration to organisms. Alternatively, the adjuvant according to the present invention, together with the virus vaccine, can be administered as a single vaccine composition.

The virus vaccine may be any virus vaccine. More specific embodiments contemplate the use of a PRRS virus vaccine such as for example Ingelvac® PRRS MLV. This is merely one exemplary PRRS virus vaccine and other such vaccines can be supplemented with the adjuvants described herein.

The immunogenic compositions described herein are particularly advantageous in the induction of the production of an antibody response to PRRS virus. In particular it is shown herein that the use of these specific adjuvants, and in particular, MCP-1, enhances immune response to PRRS virus when there is a combined administration of the adjuvant and the PRRS virus vaccine as compared to administration of vaccine alone. Such administration is shown to produce a lessening of the severity of clinical symptoms, such as lung lesions, anorexia, skin discolorations, lethargy, respiratory signs, mummified piglets, coughing, diarrhea and combinations thereof, that are associated with PRRSV infection. Indeed, there is a greater lessening of the severity of the clinical symptoms associated with PRRS virus infection observed with the combination of the vaccine and adjuvant as compared to the lessening of the severity of such symptoms produced by administration of vaccine alone in the absence of said adjuvant.

The composition thus particularly enhance the clinical outcome in a diseased animal. as compared to the outcome from administration of PRRS virus vaccine alone. In specific embodiments, the enhanced clinical outcome is a reduction of the percentage of lung lesions by at least 50% when compared to animals not receiving the immunogenic composition in combination with said adjuvant. In other embodiments, the enhance clinical outcome is a reduction of viremia in animals by at least 45% when compared to animals not receiving the immunogenic composition in combination with said adjuvant.

Thus, in one aspect, the invention relates to an improved vaccine, more particularly and improved PRRS virus vaccine, wherein the improvement comprises admixing with the virus vaccine an adjuvant selected from the group consisting of MCP-1, Haemophilus sonmus fractions, carbapol and combinations thereof.

The vaccine composition of the invention may further comprise a pharmaceutically acceptable carrier. In addition, the vaccines may comprise other active ingredients including HS, ORF 5, INF alpha, Poly ICLC, IL-12 for further enhancing the function of the PRRS vaccine. Such adjuvants may be added alone or in combination with MCP-1.

The above vaccine composition may be formulated by any method known in the art of formulation, for example, into liquid preparations, suspensions, ointments, powders, lotions, W/O emulsions, O/W emulsions, emulsions, creams, cataplasms, patches, and gels and is preferably used as medicaments. Thus, according to another aspect of the present invention, there is provided a pharmaceutical composition comprising the above vaccine composition. The vaccine composition according to the present invention, when dermally administered, can significantly induce antibody production. Accordingly, in another preferred embodiment of the present invention, the vaccine composition can be provided as a transdermal preparation.

Further, as described above, the adjuvant in the present invention may be administered, to an organism, together with the PRRS virus vaccine, as a single vaccine composition, or as an adjuvant preparation separate and distinct from the antigenic PRRS virus component of the vaccine, whereby the adjuvant acts in a manner such that amount of an antibody produced in the organism in response to the PRRS virus vaccine can be significantly increased as compared to administration of the PRRS virus vaccine alone. Thus, according to a still another aspect of the present invention, there is provided a method for increasing the amount of an antibody produced against PRRS virus, the method comprising administering an immunologically effective amount of the PRRS virus vaccine, and an adjuvant selected from the group consisting of MCP-1, Haemophilus sonmus fractions, carbapol and combinations thereof either alone or in combination with a further component selected from the group consisting of HS, ORF 5, INF alpha, Poly ICLC, IL-12 and combinations thereof, in an amount effective as an immunoadjuvant simultaneously or successively into the organism.

When the adjuvant used herein and the PRRS virus vaccine are administered to an organism, the clinical outcome of the animal is enhanced. The effective amount of the adjuvant and the immunologically effective amount of the PRRS virus vaccine may be properly determined by a person having ordinary skill in the art by taking into consideration, for example, the type and properties of the antigenic substance, the species of organisms, age, body weight, severity of diseases, the type of diseases, the time of administration, and administration method and further using the amount of an antibody produced against the antigenic substance in the organism as an index.

The PRRS virus vaccine, the adjuvant, or combinations thereof can be administered to organisms by any suitable method selected depending, for example, upon the condition of patients and properties of diseases. Examples of such methods include intraperitoneal administrationdermal administration for example, subcutaneous injection, intramuscular injection, intradermal injection, and patching, nasal administration, oral administration, mucosa administration (for example, rectal administration, vaginal administration, and corneal administration). Among them, intramuscular administration is preferred.

One embodiment of the present invention was investigated and consisted of eight groups. Groups 1, 2, 3, 4, and 5 (N=10) were inoculated intramuscularly (IM) with Ingelvac® PRRS MLV adjuvanted with different fractions of H. somnus on Day 0. Group 6 (N=10) was inoculated with Ingelvac® PRRS MLV only on Day 0. Groups 7 and 8 (N=20) were inoculated with PRRSV vaccine prototypes from a Minnesota based company, Protatek International. Group 9 (N=10) was inoculated with Ingelvac® PRRS MLV adjuvanted with MCP-1. Groups 10 (N=15) and 11 (N=5) did not receive a vaccine treatment and served as challenge and strict controls, respectively. Animals were observed daily and blood samples collected as described in Section 11.6. Animals in groups 1-10 were challenged intranasally (IN) with a virulent PRRS isolate, SDSU 73, on Day 28. Body temperatures were monitored daily following challenge. Animals were weighed on the day of vaccination, challenge, and at study termination. The study was terminated 14 days following virulent challenge (Day 42), at which time the lungs were scored for percent involvement.

As shown below in Table 1, the Hs adjuvanted prototypes seemed to perform as well as the vaccine alone, with minor differences in the clinical symptoms, and average daily weight gain and percent lung involvement, but were serologically unable to perform as well as the vaccine alone. The MCP-1 adjuvant showed the greatest reduction in lung lesions and clinical symptoms.

MCP-1 can be purified directly from peripheral blood cells or by transformed bacteria using corresponding DNA vectors. Many purification protocols are provided in the prior art. A general overview about the process and purification techniques are provided below.

The following examples set forth preferred methods and compositions in accordance with the present invention. These examples are representative in nature and nothing should be construed as a limitation upon the full scope of the invention.

### EXAMPLE 1

Peripheral Blood Mononuclear Leucocytes

Process:

MCP-1 expressing culturing lives cells derived from human glioma cell line U-105MG or human peripheral blood mononuclear leukocytes were cultured in an appropriate cell culture medium.

Purification:

Cells were separated from the cell culture and a first chromatography using a Orange A Sepharose was performed in order to solve and collect the fractions which contain the desired peptides. A second chromatography step with an appropriate cation-exchange HPLC was performed to further purify the collected fractions. A final reverse HPLC chromatography was performed in order to remove liquids and to give the purified MCP-1 peptide product as a solid.

DNA Vector Expression:

Process:

E.coli transformation with DNA vectors harbouring the MCP-1 cDNA sequences. Glycolylation is not important. Therefore a bacterial expression system is suitable.

Purification:

Procedure: Sterile filtered, Greater than 98% pure by SDS-PAGE and HPLC analyses. Endotoxin level is less than 0.1 ng per μg (1 EU/μg).

Form: Lyophilized, no additives, The protein may appear as a haze or film, which is difficult to see at the bottom of the vial.

Storage instructions: Store at +4° C. short term. Aliquot and store at −20° C. or −80° C. Avoid repeated freeze/thaw cycles.

Notes: The biological active concentration, defined its ability to chemoattract human monocytes is in a range of 10.0-100.0 ng/ml.

TABLE 1
Results Summary Table

| | | | Avg. Daily | | Virus | ELISA |
|---|---|---|---|---|---|---|
| | Clin. | ADG Post- | Rectal | | Isolation | (Avg S/P |
| | Score | Challenge | Temp | % Lung | Post-Chall. | ratio |
| Group | (Mean) | (lbs/day) | (Mean) | Involvement | (% Pos.) | Maximum) |
| PRRS + | 3.69 | 0.57 | 104.2 | 11.97 | 47.5 | 2.59 |
| WC | | | | | | |
| PRRS + | 3.88 | 0.78 | 104.1 | 8.82 | 35 | 2.38 |
| WC Sup. | | | | | | |
| PRRS + | 3.65 | 0.91 | 103.7 | 10.30 | 56.8 | 2.40 |
| LC | | | | | | |
| PRRS + | 3.89 | 0.63 | 104.1 | 19.59 | 45 | 2.63 |
| LC Sup. | | | | | | |
| PRRS + | 3.66 | 0.83 | 103.9 | 10.19 | 35 | 2.47 |
| Membranes | | | | | | |
| PRRS | 3.59 | 0.99 | 104.0 | 15.87 | 35 | 2.96 |
| MLV | | | | | | |
| Only | | | | | | |
| Protatek | 3.77 | 0.66 | 104.4 | 9.35 | 41.6 | 2.17 |
| #1 | | | | | | |
| Protatek | 3.74 | 0.66 | 104.3 | 13.31 | 52.5 | 2.50 |
| #2 | | | | | | |
| PRRS + | 3.54 | 0.69 | 104.2 | 7.42 | 25 | 2.38 |
| MCP-1 | | | | | | |
| Challenge | 4.01 | 0.32 | 104.6 | 52.71 | 85.7 | 0.99 |
| Controls | | | | | | |
| Strict | 3.00 | 1.29 | 103.5 | 0.02 | 0 | 0.06 |
| Controls | | | | | | |

In another study for the present invention, several other adjuvants and/or immunomodulators in combination with Ingelvac PRRS were evaluated for potential clinical or immunological improvements in vivo. The study evaluated Ingelvac PRRS vaccine (USDA licensed) alone and in combination with various potential immunostimulators including:
HS—H. somnus culture material which has been noted to have adjuvant activity.
ORF 5—ORF 5 peptides were obtained from 5 different PRRS isolates.
INF (Interferon) alpha DNA vaccine provided by Dr. Zuckerman along with concurrent injections of purified and recombinant INF alpha from PBL Biomedical laboratories (100,000 units/dose).
Poly ICLC (polyinosinic-polycytidylic stabilized with poly-lysine and carboxymethylcellulose) (50 ug/kg) from Ribopharm, Inc.which has been reported to act as an interferon inducer and activates anti-viral immunity.
IL-12 (interleukin 12) recombinant protein (2 ug/dose, 2 hours pre and post-vacc with MLV) from R&D Systems Inc. which has been shown to enhance T cell and NK cell activity.

The study also included an inactivated PRRSV ORF 5 peptide (0.03 mg/ml×2 ml dose) which was conjugated to cholera toxin. This CT has been reported to stimulate a better immune response due to receptor binding and antigen uptake. This was the only test article that did not include a PRRS MLV exposure.

To evaluate this objective, ninety healthy pigs at 2-3 weeks of age were obtained from a herd free of PRRS virus and screened by IDEXX serology to confirm their sero-negative status. Animals were individually ear tagged and then randomly assigned to 9 treatment groups. On day 0 of the trial (animals 3 weeks of age), animals in groups 1-8 (group 8 was placebo) received their first dose of vaccine. On study day 55, animals in groups 1-8 were exposed to a virulent challenge (PRRS 184). Animals were then necropsied on day 69 and the study terminated. The study design is detailed in the table below:

TABLE 2
Summary of Study Design

| Group | N = | Day 0 | Day 55 | Day 69 |
|---|---|---|---|---|
| 1 | 10 | PRRS Vaccine (P) | Challenge with | Necropsy |
| | | | PRRS 184 | |
| 2 | 10 | P + HS (H. somnus) | Challenge with | Necropsy |
| | | adjuvant | PRRS 184 | |
| 3 | 10 | P + PRRSV ORF5 | Challenge with | Necropsy |
| | | | PRRS 184 | |
| 4 | 10 | P + INF (interferon) alpha | Challenge with | Necropsy |
| | | | PRRS 184 | |
| 5 | 10 | P + Poly ICLC | Challenge with | Necropsy |
| | | | PRRS 184 | |
| 6 | 10 | P + IL-12 (interleukin 12) | Challenge with | Necropsy |
| | | | PRRS 184 | |
| 7 | 10 | PRRSV ORF 5 + Cholera | Challenge with | Necropsy |
| | | toxin | PRRS 184 | |
| 8 | 10 | Placebo | Challenge with | Necropsy |
| | | | PRRS 184 | |
| 9 | 10 | Strict Controls | No Treatment | Necropsy |

The primary efficacy parameter for this study was lung pathology following virulent challenge on day 55. The results of this indicate that Ingelvac PRRS vaccine alone had only 10% pneumonia following challenge compared to the virulent challenge controls which had over 36% lung involvement. In contract groups which received Ingelvac PRRS supplemented with HS or IL-12 had numerically even better protection than Ingelvac alone. Groups which received Ingelvac-ORF 5 or ORF 5-CT did not seem to have any clinical benefit and had values similar to challenge controls. Table 3 summarizes these results.

TABLE 3

| Treatment | Gross Lung Score (%) |
|---|---|
| PRRS | 10.4^{a} |
| PRRS-HS | 3.9^{a} |
| PRRS-ORF5 | 30.3 |
| PRRS-INF | 16.2 |
| PRRS-ICLC | 13.8 |
| PRRS-IL12 | 7.2^{a} |
| ORF5-CT | 45.3 |
| Chall C | 36.2 |
| Strict C | 0.42 |

^{a}Indicates significant (P < 0.05) differences between the indicated treatment groups and the challenge controls.

### EXAMPLE 3

This study consisted of eight groups. Groups 1, 2, 3, 4, and 5 (N=10) were inoculated intramuscularly (IM) with Ingelvac® PRRS MLV adjuvanted with different fractions of H. somnus on Day 0. Group 6 (N=10) was inoculated with Ingelvac® PRRS MLV only on Day 0. Groups 7 and 8 (N=20) were inoculated with PRRSV vaccine prototypes from a Minnesota based company, Protatek International. Group 9 (N=10) was inoculated with Ingelvac® PRRS MLV adjuvanted with MCP-1. (Groups 10 (N=15) and 11 (N=5) did not receive a vaccine treatment and served as challenge and strict controls, respectively. Animals were observed daily and blood samples collected as described in Section 11.6. Animals in groups 1-10 were challenged intranasally (IN) with a virulent PRRS isolate, SDSU 73, on Day 28. Body temperatures were monitored daily following challenge. Animals were weighed on the day of vaccination, challenge, and at study termination. The study was terminated 14 days following virulent challenge (Day 42), at which time the lungs were scored for percent involvement.

Table 4 provides the formulations tested for this Example:

TABLE 4
Test Article 1

| | | |
|---|---|---|
| 1. | Generic Product Name: | Ingelvac ® PRRS MLV + Hs whole cell pellet |
| 2. | Formulation: | Vaccine was a released serial formulated according to |
| | | outline of production, Hs fraction was prepared by BIVI |
| | | R&D. |
| 3. | Manufacturer: | Boehringer Ingelheim Vetmedica, Inc. |
| 3. | Storage Requirements | Long term: −70 C |
| | | short Term: 0 C |
| 4. | Assay Results: | Assay results are archived at BIVI. Titer administered |
| | | was determined by TCID₅₀ to be 5.55 logs/dose. |
| 5. | Applied Dose: | Intramuscular Injection: 2 mL, using a plastic 3 cc |
| | | syringe. |
| | | 2 mL of vaccine + adjuvant was administered via |
| | | intramuscular injection to pigs in treatment group #1 on |
| | | day of vaccination (Day 0). |

Test Article 2

| | | |
|---|---|---|
| 1. | Generic Product Name: | Ingelvac ® PRRS MLV + Hs whole cell supernatant |
| 2. | Formulation: | Vaccine was a released serial formulated according to |
| | | outline of production, Hs fraction was prepared by BIVI |
| | | R&D. |
| 3. | Manufacturer: | Boehringer Ingelheim Vetmedica, Inc. |
| 4. | Assay Results: | Assay results are archived at BIVI. Titer administered |
| | | was determined by TCID₅₀5.62 logs/dose. |

Test Article 3

| | | |
|---|---|---|
| 1. | Generic Product Name: | Ingelvac ® PRRS MLV + Hs lysed cell pellet |
| 2. | Formulation: | Vaccine was a released serial formulated according to |
| | | outline of production, Hs fraction was prepared by BIVI |
| | | R&D. |
| 3. | Manufacturer: | Boehringer Ingelheim Vetmedica, Inc. |
| 4. | Assay Results: | Assay results are archived at BIVI. Titer administered |
| | | was determined by TCID₅₀ to be 5.32 logs/dose |
| 5. | Applied Dose: | Intramuscular Injection: 2 mL, using a plastic 3 cc |
| | | syringe. |
| | | 2 mL of vaccine + adjuvant was administered via |
| | | intramuscular injection to pigs in treatment group #3 on |
| | | day of vaccination (Day 0). |

Test Article 4

| | | |
|---|---|---|
| 1. | Generic Product Name: | Ingelvac ® PRRS MLV + Hs lysed cell supernatant |
| 2. | Formulation: | Vaccine was a released serial formulated according to |
| | | outline of production, Hs fraction was prepared by BIVI |
| | | R&D. |
| 3. | Manufacturer: | Boehringer Ingelheim Vetmedica, Inc. |
| 4. | Assay Results: | Assay results are archived at BIVI. Titer administered |
| | | was determined by TCID₅₀ to be 5.27 logs/dose. |
| 5. | Applied Dose: | Intramuscular Injection: 2 mL, using a plastic 3 cc |
| | | syringe. |
| | | 2 mL of vaccine + adjuvant was administered via |
| | | intramuscular injection to pigs in treatment group #4 on |
| | | day of vaccination (Day 0). |

Test Article 5

| | | |
|---|---|---|
| 1. | Generic Product Name: | Ingelvac ® PRRS MLV + Hs membranes |
| 2. | Formulation: | Vaccine was a released serial formulated according to |
| | | outline of production, Hs fraction was prepared by BIVI |
| | | R&D. |
| 3. | Manufacturer: | Boehringer Ingelheim Vetmedica, Inc. |
| 4. | Assay Results: | Assay results are archived at BIVI. Titer administered |
| | | was determined by TCID₅₀ to be 5.13 logs/dose. |
| 5. | Applied Dose: | Intramuscular Injection: 2 mL, using a plastic 3 cc |
| | | syringe. |
| | | 2 mL of vaccine + adjuvant was administered via |
| | | intramuscular injection to pigs in treatment group #5 on |
| | | day of vaccination (Day 0). |

Test Article 6

| | | |
|---|---|---|
| 1. | Generic Product Name: | Ingelvac ® PRRS MLV |
| 2. | Formulation: | Released serial formulated according to outline of |
| | | production. |
| 3. | Manufacturer: | Boehringer Ingelheim Vetmedica, Inc. |
| 4. | Assay Results: | Assay results are archived at BIVI and included in the |
| | | final report. Titer administered was determined by |
| | | TCID₅₀ to be 5.36 logs/dose. |
| 5. | Applied Dose: | Intramuscular Injection: 2 mL, using a plastic 3 cc |
| | | syringe. |
| | | 2 mL of vaccine was administered to pigs via |
| | | intramuscular injection in treatment group #6 on day of |
| | | vaccination (Day 0). |

Test Article 7

| | | |
|---|---|---|
| 1. | Generic Product Name: | Research sample #1 of PRRS live virus vaccine |
| 2. | Formulation: | Released serial formulated according to outline of |
| | | production. |
| 3. | Manufacturer: | Protatek International, Inc. |
| 4. | Assay Results: | Assay results are archived at BIVI. Titer administered |
| | | was determined by TCID₅₀ to be 6.93 logs/dose. |
| 5. | Applied Dose: | Intramuscular Injection: 2 mL, using a plastic 3 cc |
| | | syringe. |
| | | 2 mL of vaccine was administered to pigs via |
| | | intramuscular injection in treatment group #7 on day of |
| | | vaccination (Day 0). |

Test Article 8

| | | |
|---|---|---|
| 1. | Generic Product Name: | Research sample #2 of PRRS live virus vaccine |
| 2. | Formulation: | Released serial formulated according to outline of |
| | | production. |
| 3. | Manufacturer: | Protatek International, Inc. |
| 4. | Assay Results: | Assay results are archived at BIVI. Titer administered |
| | | was determined by TCID₅₀ to be 7.58 logs/dose. |
| 5. | Applied Dose: | Intramuscular Injection: 2 mL, using a plastic 3 cc |
| | | syringe. |
| | | 2 mL of vaccine was administered to pigs via |
| | | intramuscular injection in treatment group #8 on day of |
| | | vaccination (Day 0). |

Test Article 9

| | | |
|---|---|---|
| 1. | Generic Product Name: | Ingelvac ® PRRS MLV + MCP-1 |
| 2. | Formulation: | Released serial formulated according to outline of |
| | | production. |
| 3. | Manufacturer: | Boehringer Ingelheim Vetmedica, Inc. |
| 4. | Assay Results: | Assay results are archived at BIVI. Titer administered |
| | | was determined by TCID₅₀ to be 5.42 logs/dose. |
| 5. | Applied Dose: | Intramuscular Injection: 2 mL, using a plastic 3 cc |
| | | syringe. |
| | | 2 mL of vaccine + adjuvant was administered to pigs via |
| | | intramuscular injection in treatment group #9 on day of |
| | | vaccination (Day 0). |

Challenge Treatment

| | | |
|---|---|---|
| 1) | Challenge Strain: | SDSU #73 |
| 2) | Challenge Material Preparation: | Virus was diluted 1:20 in Modified Eagles |
| | | Medium. Diluted challenge virus was to |
| | | target log 3.5 +/− 0.5 per ml. |
| 3) | Dose of Challenge Material: | 2 mL of a dilution sufficient to deliver |
| | | approximately 10⁴ logs/dose. The challenge |
| | | administration was documented on |
| | | Challenge Record Form. |
| 4) | Handling of Challenge Material: | The challenge material was diluted just prior |
| | | to the challenge procedure. The material |
| | | was kept on ice from the time of preparation |
| | | throughout the challenge procedure. |
| 5) | Testing of Challenge Material: | The titer of the challenge material was |
| | | determined by viable titration (TCID₅₀) to be |
| | | 5.3 logs/dose. |
| 6) | Method of Administration of Challenge | The challenge material was administered by |
| | Material: | intranasal inoculation. |
| 7) | Schedule of Challenge: | 28 days after vaccination |
| 8) | Frequency of Administration of | One dose of challenge material was |
| | Challenge Material: | administered to each animal on the |
| | | appropriate challenge date (Day 28). |
| 7) | Other Treatments: | None |

For randomization, a roster of all animals to be included in the study was prepared by the study investigator prior to study initiation. A computer random number generator (Microsoft Excel) was used to assign each animal a unique random number. Animals were grouped based on gender and subsequently sorted into weight blocks (experimental blocks). Within experimental blocks, the animal with the lowest random number was assigned to the first treatment group. Assignment continued by increasing random number until all animals had been assigned to a treatment group. A copy of the SOP for this randomization procedure will be placed in the study file by the study investigator.

All animals were under direct veterinary supervision for the duration of the study. Each group of pigs was housed in a different room. Pigs were monitored daily for the duration of the study. The pigs were provided commercial feed and water ad libitum throughout the study.

Table 5 sets for the inclusion criteria for the Study.

TABLE 5
Test Animal Inclusion Criteria

| | |
|---|---|
| a) Species: | Porcine |
| b) Breed: | Commercial cross |
| c) Age Requirements: | 3 weeks ± 5 days |
| d) Weight Range: | Uniform for age, range from |
| | 9.4-17.8 lbs. |
| e) Sex: | Both female and neutered males |
| f) Number: | 130 |
| g) Source of test animal and ownership: | Boehringer Ingelheim |
| | Vetmedica, Inc. |
| h) Special Requirements: | None |
| i) Serological Status: | Sero-negative to PRRS virus |

Results

Viral Titration Results

TEST ARTICLE: Ingelvac® PRRS MLV+Hs Whole Cell (per 2 ml dose):

| | | | | | | |
|---|---|---|---|---|---|---|
| BEFORE | 5.80 | 5.66 | 5.46 | 5.55 | 5.30 | 5.55 (avg) |
| INOCULATION | | | | | | |

TEST ARTICLE: Ingelvac® PRRS MLV+Hs Whole Cell Supernatant (per 2 ml dose):

| | | | | | | |
|---|---|---|---|---|---|---|
| BEFORE INOCULATION | 5.55 | 5.90 | 5.05 | 5.80 | 5.80 | 5.62 |
| | | | | | | (avg) |

TEST ARTICLE: Ingelvac® PRRS MLV+Hs Lysed Cell Pellet (per 2 ml dose):

| | | | | | | |
|---|---|---|---|---|---|---|
| BEFORE INOCULATION | 5.30 | 5.30 | 5.05 | 5.30 | 5.66 | 5.32 |
| | | | | | | (avg) |

TEST ARTICLE: Ingelvac® PRRS MLV+Lysed Cell Supernatant (per 2 ml dose):

| | | | | | | |
|---|---|---|---|---|---|---|
| BEFORE INOCULATION | 5.30 | 5.46 | 4.90 | 4.90 | 5.80 | 5.27 |
| | | | | | | (avg) |

TEST ARTICLE: Inqelvac® PRRS MLV+Membranes (per 2 ml dose):

| | | | | | | |
|---|---|---|---|---|---|---|
| BEFORE INOCULATION | 5.18 | 5.00 | 5.18 | 5.00 | 5.30 | 5.13 |
| | | | | | | (avg) |

TEST ARTICLE: Ingelvac® PRRS MLV only (per 2 ml dose):

| | | | | | | |
|---|---|---|---|---|---|---|
| BEFORE INOCULATION | 5.05 | 5.46 | 5.55 | 5.18 | 5.55 | 5.36 |
| | | | | | | (avg) |

TEST ARTICLE: Protatek PRRS Vaccine #1 (per 2 ml dose):

| | | | | | | |
|---|---|---|---|---|---|---|
| BEFORE INOCULATION | 7.30 | 6.80 | 6.80 | 6.94 | 6.80 | 6.93 |
| | | | | | | (avg) |

TEST ARTICLE: Protatek PRRS Vaccine #2 (per 2 ml dose):

| | | | | | | |
|---|---|---|---|---|---|---|
| BEFORE INOCULATION | 7.30 | 7.70 | 7.80 | 7.55 | 7.55 | 7.58 |
| | | | | | | (avg) |

TEST ARTICLE: Ingelvac® PRRS MLV+MCP-1 (per 2 ml dose):

| | | | | | | |
|---|---|---|---|---|---|---|
| BEFORE INOCULATION | 5.30 | 5.80 | 5.30 | 5.42 | 5.30 | 5.42 |
| | | | | | | (avg) |

CHALLENGE ARTICLE: SDSU #73 (per 2 ml dose):

| | | | | | | |
|---|---|---|---|---|---|---|
| BEFORE INOCULATION | 5.05 | 5.66 | 5.30 | 5.05 | 5.66 | 5.34 |
| | | | | | | (avg) |
| AFTER INOCULATION | 5.30 | 5.00 | 5.30 | 4.90 | 5.66 | 5.23 |
| | | | | | | (avg) |

Post Inoculation Weight Gain

Pigs were weighed prior to vaccination (day −7), on the day of challenge (day 28), and at necropsy (day 42). There were little differences in group average daily gains (ADG) from the days prior to vaccination to the day of challenge (day −7 to day 28) among all groups. Following virulent challenge, the challenge controls had a notably lower ADG (0.32 lbs/day) compared to all other vaccinated groups. The addition on an adjuvant did not enhance weight gain performance when compared to the vaccine alone (0.99 lbs/day). Of the adjuvanted vaccines, Ingelvac® PRRS+H.somnus Lysed Cell Pellet had the highest ADG (0.91 lbs/day), while Inglevac® PRRS+H.somnus Whole Cell had the lowest ADG (0.57 lbs/day). The live PRRS vaccines from Protatek International had only moderate weight gain with 0.66 lbs/day.

Table 6 summarizes the ADG.

TABLE 6
Average Daily Weight Gain (lbs/day)

| | | ADG from | ADG from |
|---|---|---|---|
| Group | Treatment | day −1 to 34 | day 34-55 |
| 1 | PRRS + Hs WC | 0.91 | 0.57 |
| 2 | PRRS + Hs WC Sup. | 0.91 | 0.78 |
| 3 | PRRS + Hs LC | 0.76 | 0.91 |
| 4 | PRRS + Hs LC Sup. | 0.88 | 0.63 |
| 5 | PRRS + Hs Memb. | 0.95 | 0.83 |
| 6 | PRRS MLV Only | 1.03 | 0.99 |
| 7 | Protatek #1 | 0.89 | 0.66 |
| 8 | Protatek #2 | 0.94 | 0.66 |
| 9 | PRRS + MCP-1 | 0.88 | 0.69 |
| 10 | Challenge Controls | 0.99 | 0.32 |
| 11 | Strict Controls | 1.00 | 1.29 |

Post Inoculation Clinical Observation

General observations were noted daily prior to challenge, and clinical observations were made daily following virulent challenge. All animals were generally healthy prior to challenge. Following challenge, clinical symptoms were seen in every animal that received virulent challenge, with at least 20% of the animals in each group with scores of 6 or greater (3=normal). The challenge controls had the highest mean clinical score of 4.01. Overall, all vaccinated groups were successful in reducing the severity and duration of clinical illness due to virulent challenge. Animals treated with only Ingelvac® PRRS MLV fared the best with a mean score of 3.59, while animals that received PRRS+Hs Lysed Cell Supernatant showed the most clinical illness among the vaccinates with a mean score of 3.89. The data are shown in FIG. 1.

Post Inolulation Rectal Temperatures

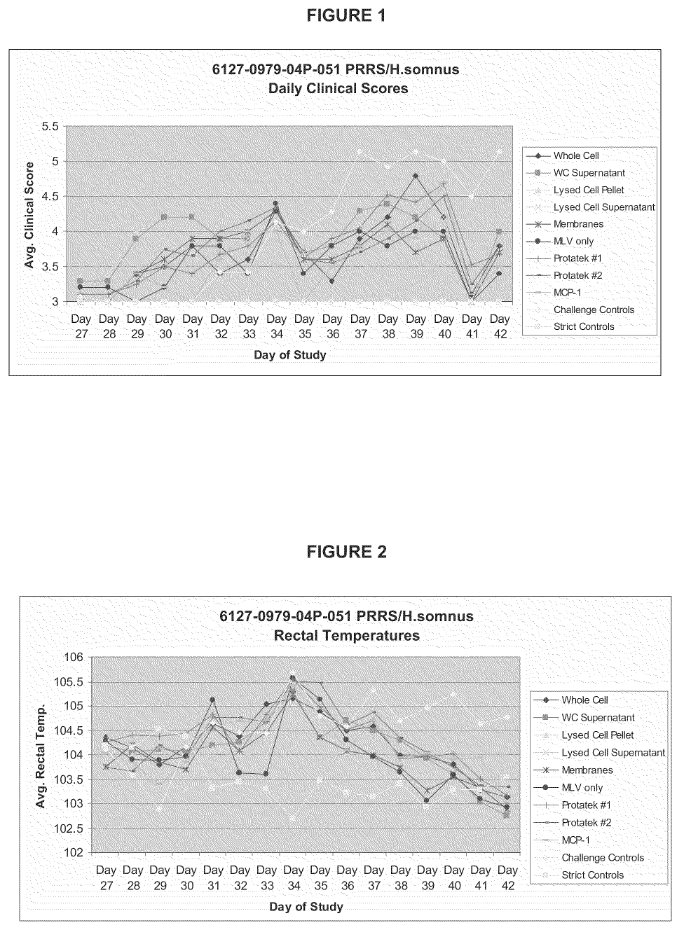

Rectal temperatures of each animal were taken daily following virulent challenge. (see FIG. 2) There were few differences seem among the groups in the first five days following virulent challenge. Rectal temperatures peaked in all challenged groups on Day 34 (5 DPC), and while the challenge controls remained elevated (Mean=104.6° F.), rectal temperatures in all of the vaccinated groups steadily declined. The group vaccinated with Ingelvac® PRRS MLV+Hs Lysed Cell Pellet had the lowest overall mean rectal temperature of 103.7° F. Table 3, Addendum 4 lists the individual lung scores.

Post Inoculation Lung Scores

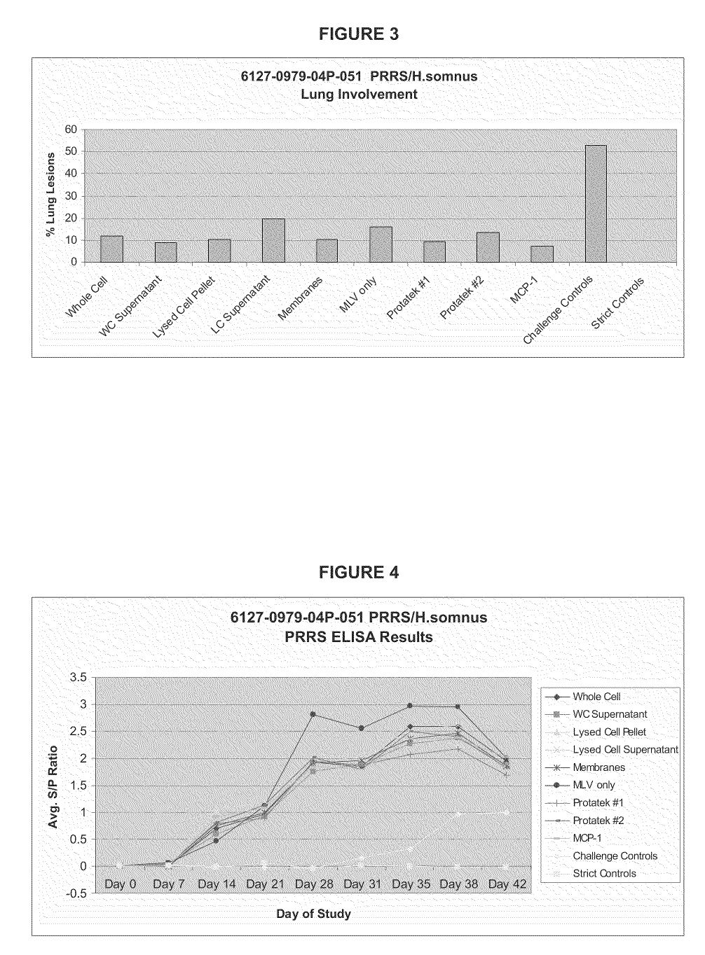

On Day 42, animals were necropsied, and the lungs of each animal were scored for percent lung consolidation related to PRRSV infection. FIG. 3 shows that all vaccinated groups showed a reduction of lung lesions when compared to the challenge controls (52.7%). The group vaccinated with Ingelvac® PRRS MLV+MCP-1 had the lowest incidence of lung involvement with and average of 7.4%. All adjuvanted vaccine groups scored lower than Ingelvac® PRRS MLV alone (15.9%), except for the group vaccinated with Ingelvac® PRRS MLV+Lysed Cell Supernatant (19.6%).

Post Inoculation Serological Status

PRRS ELISA was performed on all serum samples collected throughout the study. All vaccinated animals showed an antibody response similar to vaccination with Ingelvac® PRRS MLV alone. Following virulent challenge, antibody titers in all vaccinated groups continued to climb, but the titers from animals vaccinated with Ingelvac® PRRS MLV alone were notably higher. The results are shown in FIG. 4.

Post Inoculation Virus Isolation

Virus isolation was attempted on all samples collected throughout the study. All of the vaccinated groups were viremic following vaccination, with the percentage of positive animals gradually declining prior to challenge. Following challenge, all vaccinated groups had resurgence in viremia. All vaccinated group also showed a significant reduction in viremia by day 42 (≦15%) as compared to the challenge controls (64%).

TABLE 7
Results Summary Table

| | | | Avg. | | Virus | |
|---|---|---|---|---|---|---|
| | | | Daily | | Isolation | ELISA |
| | Clin. | ADG Post- | Rectal | | Post- | (Avg S/P |
| | Score | Challenge | Temp | % Lung | Chall. | ratio |
| Group | (Mean) | (lbs/day) | (Mean) | Involvement | (% Pos.) | Maximum) |
| PRRS + WC | 3.69 | 0.57 | 104.2 | 11.97 | 47.5 | 2.59 |
| PRRS + WC | 3.88 | 0.78 | 104.1 | 8.82 | 35 | 2.38 |
| Sup. | | | | | | |
| PRRS + LC | 3.65 | 0.91 | 103.7 | 10.30 | 56.8 | 2.40 |
| PRRS + LC | 3.89 | 0.63 | 104.1 | 19.59 | 45 | 2.63 |
| Sup. | | | | | | |
| PRRS + | 3.66 | 0.83 | 103.9 | 10.19 | 35 | 2.47 |
| Membranes | | | | | | |
| PRRS MLV | 3.59 | 0.99 | 104.0 | 15.87 | 35 | 2.96 |
| Only | | | | | | |
| Protatek #1 | 3.77 | 0.66 | 104.4 | 9.35 | 41.6 | 2.17 |
| Protatek #2 | 3.74 | 0.66 | 104.3 | 13.31 | 52.5 | 2.50 |
| PRRS + | 3.54 | 0.69 | 104.2 | 7.42 | 25 | 2.38 |
| MCP-1 | | | | | | |
| Challenge | 4.01 | 0.32 | 104.6 | 52.71 | 85.7 | 0.99 |
| Controls | | | | | | |
| Strict | 3.00 | 1.29 | 103.5 | 0.02 | 0 | 0.06 |
| Controls | | | | | | |

Discussion

The objective of this study was to evaluate several fractions of Haemophilus somnus (Hs) in combination with Ingelvac® PRRS MLV and look for potential clinical or immunological improvements in vivo when compared to the vaccine alone.

Clinically, all of the vaccinated groups performed well against the virulent SDSU #73 challenge, with reductions in clinical symptoms, rectal temperatures, and lung lesions and increased weight gains, as compared to the challenge controls. Although there were some notable differences between adjuvanted vaccine groups and the control groups, none of the experimental groups were significantly different from the animals receiving vaccine alone. There were some notable differences in lung involvement between the groups receiving the adjuvanted vaccine and the vaccine alone. All vaccinated groups showed a reduction of lung lesions when compared to the challenge controls (52.7%). Of the eight prototypes, seven prototypes had a greater reduction in lung lesions than the vaccine alone. It should be noted that the PRRS vaccine prototypes from Protatek International had virus titers of 1.5-2.0 logs higher than the prototypes using Ingelvac® PRRS MLV. It should also be noted that on Day 9, five animals from the Ingelvac® PRRS MLV only group were killed when they fell through the floor slats into the waste collection pit, which reduced the group size by 50%.

Serologically, pigs in all vaccinated groups had similar antibody responses. Following virulent challenge, antibody titers in all vaccinated groups continued to climb, but the titers from animals vaccinated with Ingelvac® PRRS MLV alone were notably higher. Virus isolation was attempted on all samples collected throughout the study, and all animals receiving the adjuvanted prototypes became viremic. By the end of the study, all vaccinated groups also showed a significant reduction in viremia (≦15%) as compared to the challenge controls (64%).

### CONCLUSION

The Hs adjuvanted prototypes seemed to perform as well as the vaccine alone, with minor differences in the clinical symptoms, and average daily weight gain and percent lung involvement, but were serologically unable to perform as well as the vaccine alone. The MCP-1 adjuvant showed the greatest reduction in lung lesions and clinical symptoms.

The PRRS vaccine prototypes from Protatek International had a greater reduction in lung lesions than Ingelvac® PRRS MLV, but were unimpressive in every other parameter.

### EXAMPLE 4

Ninety healthy pigs at 2-3 weeks of age were obtained from a herd free of PRRS virus and screened by IDEXX serology to confirm their seronegative status. Animals were individually ear tagged and then randomly assigned to 9 treatment groups. On day 0 of the trial (animals 3 weeks of age), animals in groups 1-8 (group 8 was placebo) received their first dose of vaccine. On study day 55, animals in groups 1-8 were exposed to a virulent challenge (PRRS 184). Animals were then necropsied on day 69 and the study terminated. The study design is detailed in Table 8 below:

TABLE 8
Summary of Study Design

| Group | N = | Day 0 | Day 55 | Day 69 |
|---|---|---|---|---|
| 1 | 10 | PRRS (P) | Challenge with PRRS 184 | Necropsy |
| 2 | 10 | P + HS | Challenge with PRRS 184 | Necropsy |
| | | adjuvant | | |
| 3 | 10 | P + ORF5 | Challenge with PRRS 184 | Necropsy |
| | | cocktail | | |
| 4 | 10 | P + INF alpha | Challenge with PRRS 184 | Necropsy |
| 5 | 10 | P + Poly ICLC | Challenge with PRRS 184 | Necropsy |
| 6 | 10 | P + IL-12 | Challenge with PRRS 184 | Necropsy |
| 7 | 10 | ORF 5 + | Challenge with PRRS 184 | Necropsy |
| | | Cholera toxin | | |
| 8 | 10 | Placebo | Challenge with PRRS 184 | Necropsy |
| 9 | 10 | Strict Controls | No Treatment | Necropsy |

Results

PRRS Serology—IDEXX ELISA and Serum Neutralization

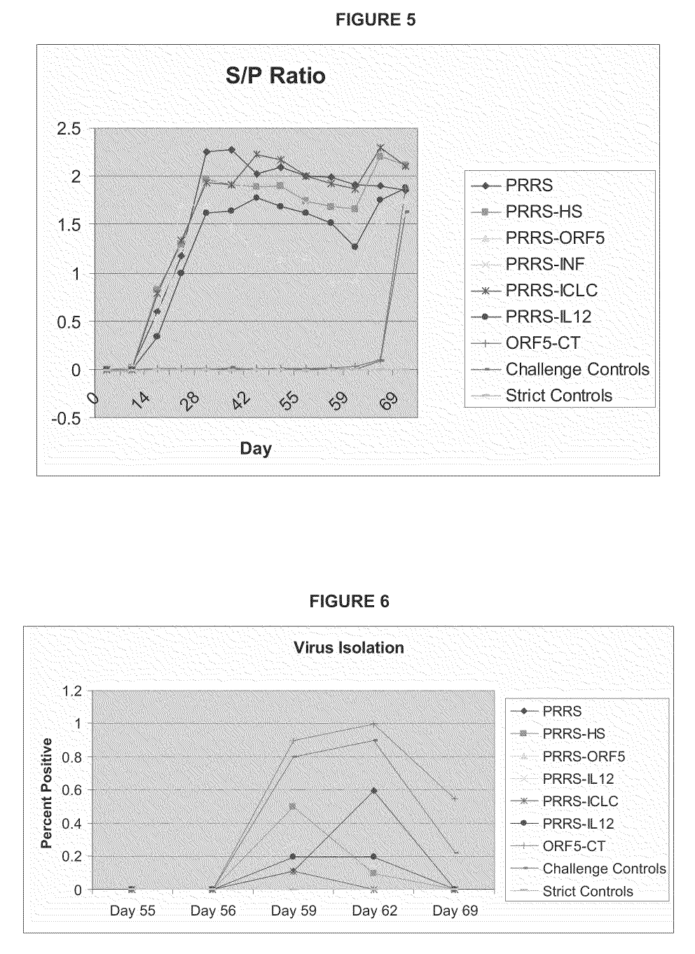

The serum collected on days 0, 7, 14, 28, 35, 42, 49, 55, 56, 59, 62, and 69 were tested in the IDEXX PRRS ELISA to monitor seroconversion. All groups that had Ingelvac PRRS exposure on day 0 (Group 1-6) had detectable seroconversion by day 14 and then were positive for the duration of the study. Although the significance is not known or understood, treatment group 5 (PRRS+ORF 5) had the lowest S/P ratios of the group. Groups 7 and 8 remained seronegative until after virulent challenge exposure. Group 9 remained seronegative for the duration of the trial and thereby confirmed study validity. See FIG. 5 for further details.

On days 0, 14, 28, 42, 55, and 69, serum samples were also tested for serum neutralizing activity to two different PRRS isolates. It is known that the isolate used in the SN test can impact the end result and is generally accepted that the more homologous, the higher the potential SN. In this study, two different heterologous isolates were used in the SN assay to see if any treatment expanded/extended the induction of SN antibodies. The results of SN testing against PRRS isolates SDSU 73 and MN 184 is shown in Table 9.1:1 below. Prior to day 55, there were very few SN antibodies detected (<4). On day 69, which was 14 days post-challenge, neutralizing titers were detected in several of the treatment groups.

Using the SDSU 73 PRRS isolate, the PRRS and PRRS-HS had significantly (P<0.05) higher levels than the PRRS-INF and PRRS-INF and PRRS-IL12 groups, respectively. Using the MN 184 virus, the PRRS treatment group had significantly (P<0.05) higher titers than PRRS-INF, ORF5-CT, and challenge control groups. The PRRS-ORF5 had significantly (P<0.05) higher titers than the ORF5-CT and challenge control groups. See

Table 9 below for group average values.

TABLE 9
Average SN titers (GMT) using 2 different neutralizing
virues.

| | | SN MN 184 |
|---|---|---|
| Treatment | SN SDSU 73 (GMT) | (GMT) |
| PRRS | 9.2^{a} | 18.4^{c} |
| PRRS-HS | 14.9^{b} | 9.7 |
| PRRS-ORF5 | 4.3 | 13.1^{d} |
| PRRS-INF alpha | 1.3^{ab} | 2.1^{c} |
| PRRS-Poly ICLC | 4 | 4.8 |
| PRRS-IL2 | 2.5^{b} | 5.9 |
| ORF5-CT | 0 | 1.3^{cd} |
| Chall Controls | 0 | 1.1^{cd} |
| Strict Controls | 0 | 0 |

^{a}= significant difference between PRRS and PRRS-INF alpha
^{b}= significant difference between PRRS-HS and PRRS-INF alpha and PRRS-IL12
^{c}= significant difference between PRRS and PRRS-INF alpha, ORF 5-CT, and challenge controls
^{d}= significant difference between PRRS-ORF5 and ORF5-CT and challenge controls.

Virus Isolation

The serum samples collected were placed on MA-104 cells and incubated for 8 days. After this time, samples were evaluated for CPE typical of PRRS infection and scored as PRRS positive or PRRS negative. The proportion of positive pigs in the challenge control group was significantly (P<0.05) higher than the PRRS, PRRS-INF alpha, and PRRS-ICLC on day 59. The proportion of positive pigs in the challenge control group was significantly (P<0.05) higher than the PRRS-HS, PRRS-ORF5, PRRS-INF, PRRS-ICLC, and PRRS-IL12 on day 62. There were no significant differences on day 69. See FIG. 6 for percentages positive.

Weights

Animals were weighed on day 0, day 55 (virulent challenge), and day 69 (necropsy, study termination). Animals were randomized to treatment group on day 0 and there were no significant differences between treatments at the start of the study. On day 56, the PRRS and PRRS-HS had significantly higher average group weights than did the PRRS-ORF 5 treatment group. On days 55 and 69, the ORF5-CT was significantly lower than all other treatment groups. Data is summarized below in Table 10

TABLE 10
Average group weights (lbs)

| | Day 0^{a} | Day 42 | Day 55 | Day 69 |
|---|---|---|---|---|
| PRRS | 15.5 | 15.9 | 97.9^{b} | 105.1 |
| PRRS-HS | 15.1 | 15.6 | 93.6^{b} | 106.2 |
| PRRS-ORF5 | 15.3 | 16.0 | 68.5^{bc} | 84.2^{c} |
| PRRS-INF | 15.5 | 16.2 | 96.1 | 113.9 |
| PRRS-ICLC | 15.6 | 16.3 | 90.4 | 105.5 |
| PRRS-IL12 | 15.4 | 16.3 | 93.7 | 109.6 |
| ORF5-CT | 15.2 | 15.8 | 101.5 | 107.8 |
| Chall Control | 15.4 | 16.1 | 100.8 | 113.1 |
| Strict Control | 15.4 | 16.0 | 90.8 | 112.3 |

^{a}= no significant difference on day 0 of the trial
^{b}= Significant differences between PRRS and PRRS-HS and the PRRS-ORF5
^{c}= PRRS-ORF5 was significantly lower than all treatment groups at this time period.

Clinical Observations

Animals were monitored daily following virulent challenge. During the trial few clinical signs were noted and most groups were noted as normal (score of 3) on a daily basis.

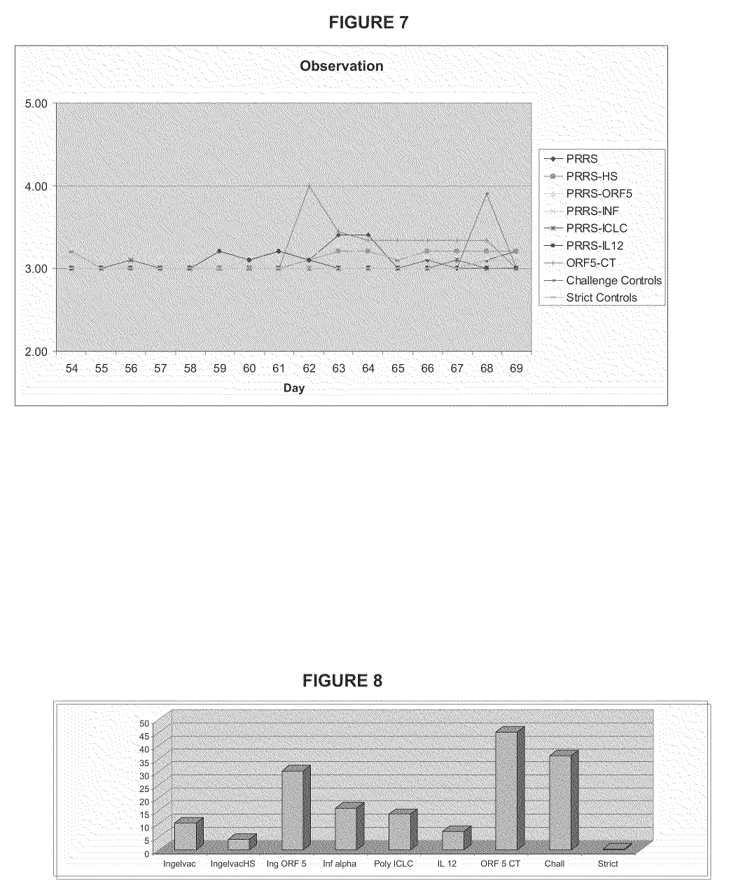

There were few clinical signs noted in all groups except for groups 7 (ORF5-CT) and 8 (challenge controls) and no statistics were done on this parameter. See FIG. 7 for average daily clinical score.

Lung Pathology and IHC

Animals were necropsied 14 days post challenge and had their lungs scored for gross lung pathology associated with PRRS. The average group scores are shown in FIG. 8. The PRRS, PRRS-HS, and PRRS-IL12 all had significantly (P<0.05) lower average gross lung pathology than the challenge control group. The ORF5-CT group was also significantly (P<0.05) higher than all other treatment groups.

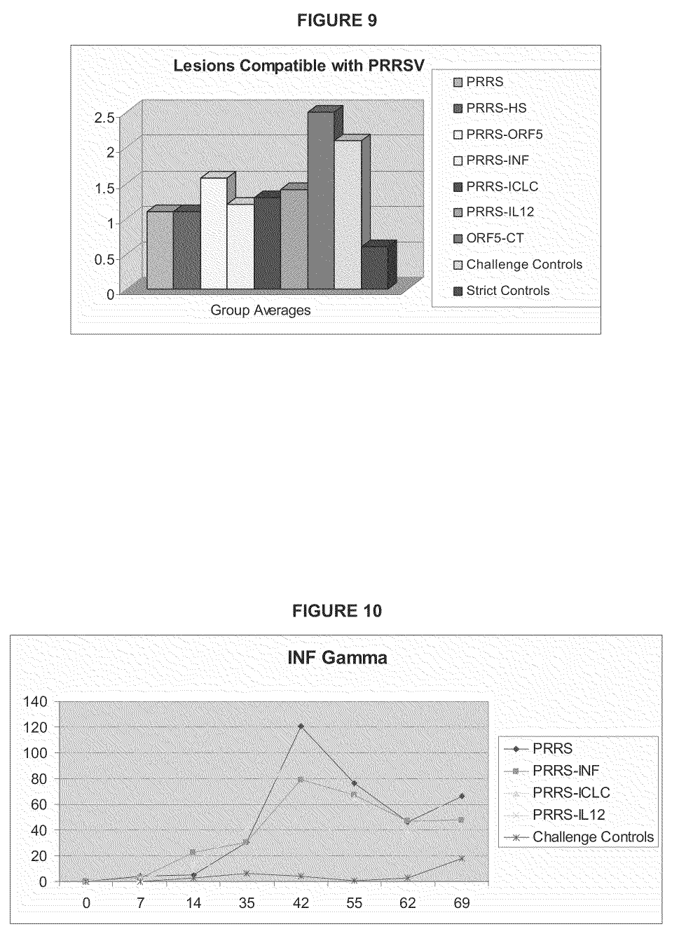

The second evaluation that further supports the gross lung pathology is the lesion score as measured by IHC. Based on this data, all groups except the PRRS-ORF5 and ORF5-CT were significantly (P<0.05) lower than the challenge control group (FIG. 9).

Immunology

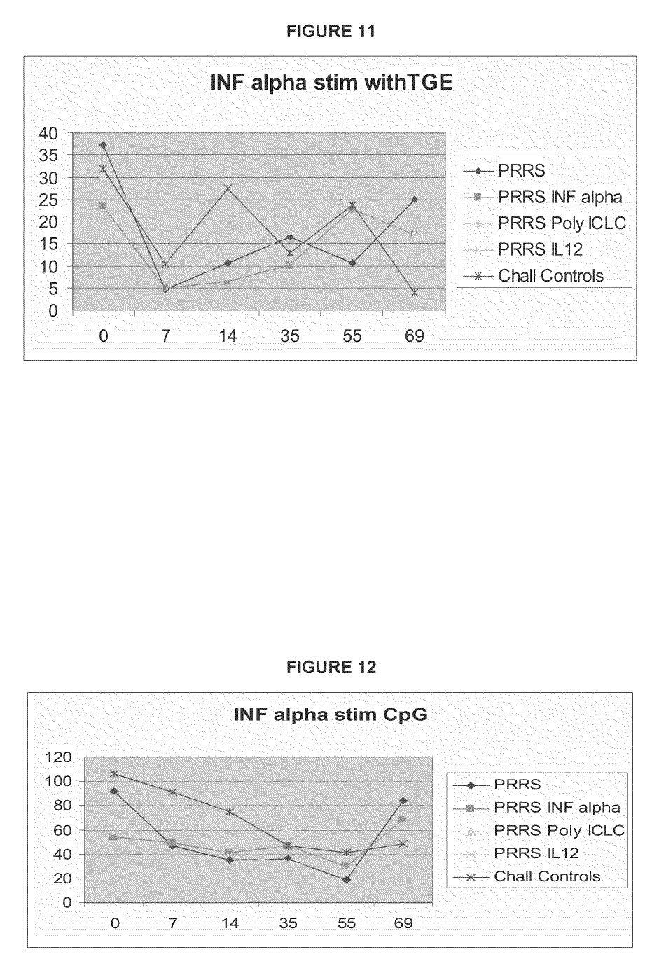

Lymphocyte samples were collected and sent to Federico Zuckerman for INF gamma ELI-SPOT and INF-alpha ELI-SPOT. Results of INF gamma are shown in FIG. 10 below. The INF alpha response was evaluated using both TGE (FIG. 11) and CpG (FIG. 12) stimulation.

The INF gamma measurements peaked after vaccination, with the PRRS vaccine alone and PRRS-ICLC being the highest. There was tremendous variation within groups in terms of animal response and values quickly declined and were equivalent between groups.

The INF alpha data was highest on day 0 and then had a slight peak again on the final bleed date. PRRS alone and PRRS-ICLC were again the highest.

Discussion

This study was conducted in an attempt to use PRRS MLV vaccines and then further modulate the immune response to improve overall protection and clinical efficacy. The primary parameter of vaccine efficacy is gross lung pathology following heterologous challenge.

In this study, the PRRS vaccine, PRRS-HS, and PRRS-IL12 all had significant reductions of gross lung lesions compared to the challenge controls. None of these 3 vaccines were different from each other. The ORF5-CT vaccine prototype has the highest lung lesions and further confirmed that inactivated vaccines not only don't provide efficacy but may even exacerbate lesions.

Virus isolation following virulent challenge indicated that viremia peaked on days 5 and 8 post-challenge. The challenge controls and ORF5-CT had the highest levels of viremia with over 80% of the animal positive at both time points. In contrast, on day 5 the PRRS, PRRS-INF, and PRRS-ICLC all had significantly (P<0.05) lower levels of virus than did the challenge controls. On day 8 post-challenge, the PRRS-HS, PRRS-ORF5, PRRS-INF, PRRS-ICLC, and PRRS-IL12 all had significantly (P<0.05) lower levels of viremia than did the challenge controls. Interestingly, the PRRS MLV alone was not significant and so addition of these test components may aid in virus reduction and clearance.

The serological response in this study was measured by both IDEXX ELISA and SN assay. The IDEXX response was predictable and all vaccines that included an MLV vaccine component seroconverted by day 14 and stayed positive for the duration of the trial. The relevance of the quantitative S/P ratio values is questionable, but it is interesting to note that PRRS-ORF5 was the lowest MLV-containing vaccine and the ORF5-CT (inactivated) vaccine induced no immune response until after virulent challenge exposure.

This trial also looked at SN titers using 2 different viruses (SDSU 73 and MN 184) as the neutralization target. It was hypothesized that some level of immunmodulation may broaden the immune response. All groups remained SN negative until after virulent challenge and titers were only detected on day 69, which was the last day of the trial. If SN titers would have continued to climb is unknown, but would be interesting to monitor in future studies. Using the SDSU 73 virus, the PRRS ( 1/9) and PRRS-HS ( 1/15) groups had the highest reported SN titers and were significantly (P<0.05) different than some other treatment groups. Using the Minn 184 virus, the PRRS ( 1/18), PRRS-HS ( 1/10), and PRRS-ORF 5 ( 1/13) had numerically the highest SN titers. The PRRS and PRRS-ORF5 were significantly (P<0.05) different from some of the groups in the trial. It was interesting to note that PRRS-HS had the highest titer with SDSU 73 but not with the Minn 184, although it was relatively higher in both groups.

Evaluation of weights showed that all treatment groups started out at equivalent starting weights in the trial, but that the PRRS-ORF 5 group was significantly lower than PRRS and PRRS-HS by day 56 and lower than all treatment groups by day 69.

This was a complex study and other than the commercially available vaccine, no other prototype excelled in every parameter measured. In order to further assess this, a qualitative table (Table 11) shown below, ranks prototypes for the study parameters.

TABLE 11

| | | | | | INF | INF | | |
|---|---|---|---|---|---|---|---|---|
| Treatment | Serology/SN | VI | Weight | Pneum | alpha | gamma | Score | Rank |
| PRRS | Pos | Pos | Pos | Pos | Pos | Pos | 6/6 | 1 |
| PRRS-HS | Pos | Pos | Pos | Pos | — | — | 4/6 | 2 |
| PRRS- | Pos | Pos | Neg | — | — | — | 1/6 | 5 |
| ORF5 | | | | | | | | |
| PRRS-INF | — | Pos | — | — | — | — | 1/6 | 5 |
| PRRS-ICLC | — | Pos | — | — | Pos | Pos | 3/6 | 3 |
| PRRS-IL12 | — | Pos | — | Pos | — | — | 2/6 | 4 |
| ORF5-CT | Neg | Neg | — | Neg | — | — | −3 | DEAD |
| Chall | — | — | — | — | — | — | — | — |
| Controls | | | | | | | | |
| Strict | — | — | — | — | — | — | — | — |
| Controls | | | | | | | | |

### CONCLUSION

Ingelvac PRRS is a solid tool for use in controlling clinical disease caused by PRRS both in a research trial and under field conditions. Use of adjuvants and immunostimulants has the potential to further enhance this already efficacious product.

1. An immunogenic composition comprising a virus vaccine and an adjuvant selected from the group consisting of MCP-1, Haemophilus sonmus fractions, carbapol and combinations thereof.

2. The immunogenic composition of claim 1, wherein with the virus vaccine is comprised of a PRRS virus.

3. The immunogenic composition of claim 2, wherein said PRRS virus is a recombinant PRRS virus or an attenuated live virus.

4. The immunogenic composition of claim 1, wherein said virus vaccine is Ingelvac® PRRS MLV.

5. The immunogenic composition of claim 1, wherein said adjuvant is MCP-1 and said virus vaccine is a modified live virus.

6. A method of enhancing immune response to PRRS virus comprising the steps of administering to pig an effective amount of:a) an adjuvant selected from the group consisting of MCP-1, Haemophilus sonmus fractions, carbapol and combinations, and b) a virus vaccine, wherein the combined administration of said adjuvant and said PRRS virus vaccine produces an enhanced immune response to the PRRS virus as compared to administration of vaccine alone.

7. The method of claim 6, wherein the adjuvant and the PRRSV vaccine are administered simultaneously.

8. The method of claim 6, wherein the adjuvant and the PRRSV vaccine are administered sequentially.

9. The method of claim 6, wherein the adjuvant and/or the vaccine is administered mucosally.

10. The method of claim 9, wherein the adjuvant and/or the vaccine is administered via a route selected from the group consisting of intranasal, ocular, gastrointestinal, oral, rectal and genitourinary tract.

11. The method of claim 10, wherein the adjuvant and/or the vaccine is administered intranasally.

12. The method of claim 10, wherein the adjuvant and/or the vaccine is administered parentally.

13. The method of claim 12, wherein the adjuvant and/or the vaccine is administered via a route selected from the group consisting of intraperitoneal, intravenous, subcutaneous or intramuscular.

14. A method for lessening the severity of clinical symptoms associated with PRRSV infection comprising the step of administering a composition of claim 1.

15. The method of claim 14, wherein the clinical symptoms are selected from the group consisting of lung lesions, anorexia, skin discolorations, lethargy, respiratory signs, mummified piglets, coughing, diarrhea and combinations thereof.

16. The method of claim 14, wherein administration of a composition of claim 1 produced a greater lessening of the severity of the clinical symptoms associated with PRRS virus infection as compared to the lessening of the severity of such symptoms produced by administration of vaccine alone in the absence of said adjuvant.

17. A method of producing an enhanced clinical outcome in an animal having a PRRS virus infection, comprising administering to said animal a composition of claim 2, wherein said clinical outcome is enhanced as compared to the outcome from administration of PRRS virus vaccine alone.

18. The method of claim 17, wherein said enhanced clinical outcome is a reduction of the percentage of lung lesions by at least 50% when compared to animals not receiving the immunogenic composition in combination with said adjuvant.

19. The method of claim 17, wherein said enhanced clinical outcome is a reduction of viremia in animals by at least 45% when compared to animals not receiving the immunogenic composition in combination with said adjuvant.

20. An improved PRRS virus vaccine composition, said improvement comprising admixing with said PRRS virus vaccine an adjuvant selected from the group consisting of MCP-1, Haemophilus sonmus fractions, carbapol and combinations thereof.
